(19) United States
(12) Patent Application Publication
(10) Pub. No.: US 2020/0123589 A1
(43) Pub. Date: Apr. 23, 2020

(54) GALACTOSE RAPID QUANTITATIVE DETECTION SYSTEM AND USE THEREOF

(71) Applicant: Avalon HepaPOC Limited, Hong Kong (CN)

(72) Inventors: Oliver Yoa-Pu HU, Taipei City (TW); Sz-Hau CHEN, Hsinchu City (TW); Ping Yang, Taipei City (TW); Hsin-Ju LIN, Hsinchu City (TW); Po-Yuan TSENG, Hsinchu City (TW); Thomas Y.S. SHEN, Hsinchu City (TW); Johnson Yiu-Nam LAU, Hong Kong (CN); Ching-Yuan CHU, Hsinchu City (TW)

(21) Appl. No.: 16/165,722

(22) Filed: Oct. 19, 2018

(51) Int. Cl. C12Q 1/54; G01N 33/50; G01N 35/00

(52) U.S. Cl. CPC C12Q 1/54; G01N 33/5088; G01N 35/00029; G01N 2035/00891; G01N 2800/042; G01N 2035/00108; G01N 35/00722

(57) ABSTRACT

A galactose rapid detection system has a galactose composition including a galactose, a buffer solution and an 0-99% antioxidant, which enters a human body after metabolism and produces a biological sample; a test strip or a filter paper, comprising an enzyme, the enzyme would react with the biological sample producing a electrochemical information ;a meter including a power supply unit for providing a signal; a connector for receiving the signal provided by the power supply unit, transmitting the signal to the test strip or the filter paper, wherein the signal reacting with the electrochemical information produce a corresponding response signal, and the connector transmit the corresponding response signal to the meter; a calculation unit for calculating the corresponding response signal; an A/D convertor for receiving the corresponding response signal from the calculation unit, transforming the corresponding response signal into a digital reaction signal calculated by the calculation unit; and a processor for processing the digital reaction signal a display for displaying the digital reaction signal; and a digital terminal for receiving the digital reaction signal.

### FIELD OF THE INVENTION

The invention relates to a galactose detection system, particularly to rapidly measuring galactose concentration in biological sample and evaluating the impairment degree of liver functions.

### BACKGROUND OF THE INVENTION

The liver is closely related to the clearance of many drugs which can be cleared via different metabolic pathways or via bile excretion. Changes in the rate of excretion or metabolism of a drug caused by abnormal liver functions may cause the drug to accumulate or inhibit the formation of active metabolites. Galactose in blood is sensitively correlated with abnormal liver functions and, from research literature, evidence shows that the galactose value in blood is significantly related to the impairment degree of liver functions. Therefore, the residual functions of the abnormal liver can be evaluated according to the galactose value in blood.

The conventional detection method is used for intravenous injection of 0.5 g/kg galactose after fasting for 8 hours, and measured the galactose concentration in plasma after 60 minutes (Tang H. S. et al. (1992) Digestion, 52:222-231; Ranek L. et al. (1983) Clin. Physiol. 3:173-178). The measurement method comprises: drawing a measurement curve according to a relationship between different concentrations of standard galactose solutions and light absorption values thereof; adding HClO₄ in the extracted blood and shaking for mixing, then taking supernatant by centrifugation; adding KOH into the supernatant and shaking for mixing, then taking supernatant by centrifugation again; and then adding galactose dehydrogenase into the supernatant and placing in a dark room for 60 minutes to avoid color reaction inaccuracy for preparing of a specimen and measuring the light absorption value thereof; and finally finding the concentration value by the measurement curve. However, the detection process is complicated and time-consuming, and requires using a variety of medicaments. Therefore, it takes lengthy procedure to learn the detecting result.

Taiwan patent No. I292478 disclosed a method of making the test specimen for the determination of liver function and sampling test strip. The method also needs to be injected with galactose into the body of a subject, and waits for 60 minutes to measure the concentration of galactose in blood. The measurement method comprises: drawing a measurement curve according to a relationship between different concentrations of standard galactose solutions and light absorption values thereof; adding trichloroacetic acid to the test paper and shaking for 30 minutes, then taking the solvent out and adding a solvent containing galactose dehydrogenase therein and shaking for 30 minutes, and then adding a chromogenic agent into the resulting solvent, and finally measuring the light absorption value thereof. However, the method is based on galactose injection into the human body and needs to make the test specimen. The detection process is complicated and time-consuming. Therefore, a rapid and simple galactose detection method is required in the art for patients who need to detect galactose.

Taiwan patent M488635 disclosed the biological test strip; U.S. Pat. No. 971,995 disclosed the system of detecting hematocrit test, the system comprising an electrochemical test strip and a meter. Due to the above prior arts, it is a very common technology of supervising body condition by electrochemical method. It is because the instability of the enzyme protein, the enzyme cannot be preserved in an alkaline environment or dry condition. The enzyme is thereby generally stored in an acidic solution, such as preserved in acidic amine sulphate solution with a very short storage time. Therefore, providing a test strip which can be stored in solid state for a long time is another problem to be solved in the field.

### SUMMARY OF THE INVENTION

An object of the present invention is to provide a galactose rapid quantitative detection system, comprising a galactose composition, a test strip or a filter paper and a meter.

The galactose composition includes a galactose, a buffer, and an 0-99% antioxidant, which enters a human body after metabolism and produces a biological sample;

The test strip or the filter paper, comprising an enzyme, the enzyme would react with the biological sample producing a electrochemical information; and

The meter includes a power supply unit for providing a signal. The connector is used to receive the signal provided by the power supply unit and transmit the signal to the test strip or the filter paper, wherein the signal reacting with the electrochemical information produce a corresponding response signal, and the connector transmit the corresponding response signal to the meter. The calculation unit is used to calculate the corresponding response signal. The A/D convertor is used to receive the corresponding response signal from the calculation unit, transforming the corresponding response signal calculated by the calculation unit into a digital reaction signal. The processor is used to process the digital reaction signal. The display for displaying the digital reaction signal and a digital terminal for receiving the digital reaction signal

To achieve the object above, the buffer is selected from a group including acetic buffer, citrate buffer, phosphate buffer, acetate buffer, carbonate buffer, ascorbic acid buffer, and triethanolamine buffer.

To achieve the object above, the antioxidant is selected from the group including vitamin C or/and sodium bisulfite, vitamin A, vitamin E, flavonoids, polyphenols, Ethylenediaminetetraacetic acid(EDTA), Diethylenetriaminepentaacetic acid (DTPA), and NTA-Nitrilotriacetate acid (NTA).

To achieve the object above, the galactose including D-(+)-galactose, L-(−)-galactose, stable isotope galactose, cyclic galactose or galactose derivative.

To achieve the object above, the galactose composition is administrated through oral administration, injection, spray, inhalation, buccal, rectal, suppository or other medical acceptable way.

To achieve the object above, the way of oral administration is to let users take the galactose composition in advance, then the content of galactose in the human body is measured by measuring the content of galactose in the biological sample.

To achieve the object above, the way of injection is to let users inject the galactose composition into the body in advance, then the content of galactose in the biological sample of the body is measured by measuring the content of galactose in the biological sample.

Another object of the present invention is to provide a test strip, the test strip comprises an insulating substrate, an electrode unit configured on the insulating substrate, and a first insulating spacer covering a part of the electrode unit and including a reaction zone channel sited at a first edge of the insulating spacer, wherein another part of the electrode unit is exposed to the reaction zone channel; and

a second insulating spacer including a second edge, the second insulating spacer covering the reaction zone channel of the first insulating spacer and the first edge of the first insulating spacer , the second edge of the second insulating spacer , and the same side edge of the insulating substrate are all in a convex arc shape, and the edge of the insulating substrate concaves inwards relative to the front half part of the reaction zone channel ; wherein the reaction zone channel comprises at least a reaction layer, the reaction layer is covered by the electrode unit in the reaction zone channel including at least galactose and a conductive medium to react with the biological sample through electrochemical reaction; wherein the test strip utilizes the convex tip of the second edge of the second insulating spacer and the concave structure of the insulating substrate relative to the front half part of the reaction zone channel to reduce the cohesive force of the biological sample, and enables the biological sample to go forward rapidly under the action of capillary phenomenon; wherein the enzymes can oxidize, reduce, decompose, or metabolize galactose.

To achieve the object above, the test range of galactose in the test strip is 50-2000 μg/ml.

To achieve the object above, the insulating substrate is selected from the group consisting of polyvinyl chloride (PVC), glass fiber (FR-4), polyester suphone, bakelite plate, polyethylene terephthalate (PET), polycarbonate (PC), polypropylene (PP), polyethylene (PE), polystyrene (PS), glass plate, ceramic or any combination thereof.

To achieve the object above, wherein the electrode unit is selected from the group consisting of palladium, platinum, gold colloid, titanium, carbon, silver, copper, gold and silver.

To achieve the object above, the reaction layer is selected from the group consisting of enzyme, coenzyme, buffer solution, stabilizer and surfactant.

To achieve the object above, the conductive medium is selected from the group consisting of ferrocene, ferrocenium, methylene blue, tris(acetonitrile)ruthenium trichloride, dihydroxybenzoquinone, phenazinemethosulfate, tetrathiafulvalene tetra-cyano-quino-dimethane, methyl viologen, toluidine blue, 5,6-diamino-1,10-phenanthroline, 2,2′-bipyridine.

To achieve the object above, the conductive medium further compries metal ion compound, the metal ion compound is selected from the group consisting of MgCl₂, BeCl₂, CaCl₂, SrCl₂, BaCl₂ and any one combination thereof.

To achieve the object above, the buffer solution is selected from the group consisting of Tris, Tris-HCl, PBS, MES, CHES, Borate, Universal buffer mixtures (CPB), MOPS, TES, HEPES, TAPSO, Tricine, Bicine and TAPS.

To achieve the object above, the stabilizer is selected from the group consisting of Xylitol, mannitol, polyxylose, araboxylan, mannan, trehalose, PEG, PVA, PEO, Methocel, agarose, sol-gel, collagen, chitosan, BSA, casein, neo protein, amino acid and any one combination thereof.

To achieve the object above, the surfactant is selected from the group consisting of a cationic surfactant, an anionic surfactant, a neutral ionic surfactant, and a nonionic surfactant.

To achieve the object above, the enzyme can be dried, solidified and stored in a neutral, acidic or alkaline environment

Another object of the present invention is to provide a method of performing the galactose rapid quantitative detection system within a user, comprising:
(1) The user take into a galactose composition in advance;
(2) A biological sample is obtained by using a biological sampling pen;
(3) the biological sample is absorbed by a test strip from the biological sampling pen;
(4) the test strip is inserted into a meter ; and
(5) the user or a professional medical staff read the value of galactose concentration to determine a disease or liver residual function of the user.

To achieve the object above, the method can be manipulated by the subject or professional staff.

To achieve the object above, the disease is neonatal galactosemia.

### BRIEF DESCRIPTION OF THE DRAWINGS

FIG. 1 is a frontal appearance view of a galactose rapid detection system.

FIG. 2 is a block schematic view of a meter system in the galactose rapid detection system.

FIG. 3 is an accuracy test result for the galactose rapid detection system.

FIG. 4 is a precision test result for the galactose rapid detection system.

FIG. 5 is a schematic view of the test strip structure.

FIG. 6 is a blood volume analysis of a general filter paper.

FIG. 7 is an accuracy test result of galactose under various volumes of the test strip.

FIG. 8 is a test result for the test strip storage days.

FIG. 9 is a test result for hematocrit evaluation tests.

FIG. 10 is a test result for repeatability evaluation tests.

FIG. 11 is one kind of correlation between intravenous injection galactose GSP result and oral administration galactose OGSP result.

FIG. 12 is another kind of correlation between intravenous injection galactose GSP result and oral administration galactose OGSP result.

FIG. 13 is a test strip detection result completed by a semiautomatic robotic arm.

### DETAILED DESCRIPTION OF THE PREFERRED EMBODIMENTS

The present invention is exemplified in the following embodiments, but is not limited thereby. Otherwise stated, the materials used in the present invention are all available on market.

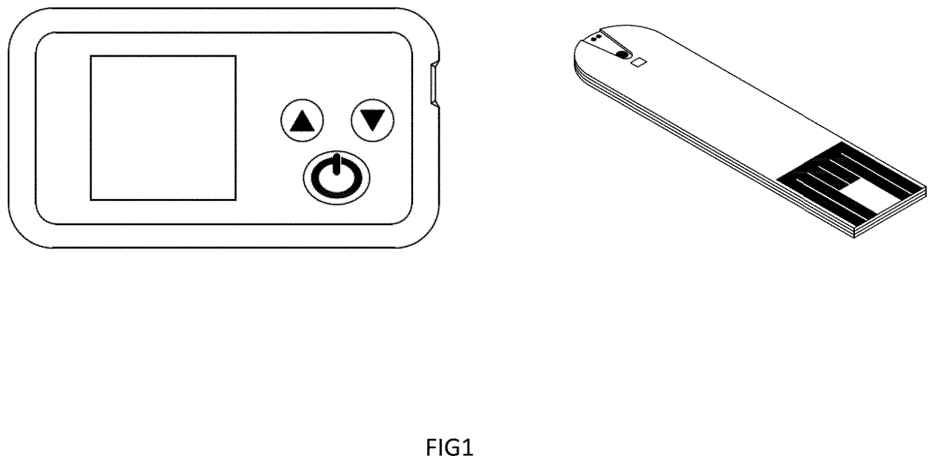

The galactose rapid detection system shown in FIG. 1a of the present invention adopts the enzyme electrochemical sensing technology. The system mainly adopts a disposable dry enzyme electrode instrument technology, utilizes the galactose or metabolites thereof which are metabolized by liver in human body reacting with enzyme to generate micro-current through electrochemical reaction, then the reading value of galactose is detected by measuring the micro-current. The residual liver function is evaluated according to the reading value. The galactose rapid quantitative detection system of the present invention is not limited to the evaluation of liver function, but also can diagnose diseases related to galactose, such as neonatal galactosemia. Furthermore, the galactose of the present invention further comprises galactose and its derivatives. The biological sample can be blood, saliva, urine, lavage, or any other bodily fluid.

### Embodiment 1: Method for the Use of the Galactose Rapid Quantitative Detection System

1-1 The Use of Test Strip for Galactose Detection

The galactose test strip shown in FIG. 1b is encapsulated in an aluminum foil bag, and stored at 4° C.-10° C. (39.2° F.-51.2° F.) temperatures. Before use, the test strip needs to be warmed for 20 minutes. After being unsealed, the galactose test strip needs to be used in 30 minutes. Once this time has passed, the test strip is discarded and not allowed to be used again.

1-2 Sample Acquisition and Preparation

The user needs to drink the oral galactose composition first, wherein the content of the galactose is 1%-80%, preferably 4%-40%, by weight of the total galactose composition, wherein the buffer solution can be not added or can be added to the total weight of 0.001%-5%, and the antioxidant can be not added or can be added to the total weight of 0.001%-5%. Appropriate formula can be prepared by selecting the buffer and the antioxidant, and adding the content of the following ingredients: antioxidant of 0.01M-1M selected from a group including vitamin C, sodium bisulfite, vitamin A, vitamin E, flavonoids, polyphenols, Ethylenediaminetetraacetic acid (EDTA), Diethylenetriaminepentaacetic acid (DTPA), and NTA-Nitrilotriacetate acid (NTA); and/or buffer solution of 0.01M-1M selected from a group including acetic buffer, citrate buffer, phosphate buffer, acetate buffer, carbonate buffer, ascorbic acid buffer, and triethanolamine buffer with adjustment of the pH values ranged 4.0-9.0. A stable formula can be obtained by adding 0.01% citrate buffer and 0.5% sodium bisulfite with a pH value of 4.5. After drinking the above galactose composition for 60 minutes, fingers are cleaned with soap and warm water and wiped dry, then fingertips are wiped with alcohol cotton before biological sampling. After the fingertips are completely dry, biological samples are obtained by using a biological sampling device to lightly prick the fingertips, and should avoid excessive squeezing during the biological sampling part.

1-3 Use Procedure

(1) Password Card Calibration

In order to measure a correct galactose value, the galactose meter should be re-calibrated when a new box of galactose test strip is used every time. When in calibration, only the password card attached on the box is allowed to be used, and confirm that the password of the password card is the same as the password on the test strip box used for galactose detection; then insert the contact electrode of the password card into a password card slot of the galactose meter. After inserting the galactose test strip into the test strip slot of the meter, the meter will automatically activated and show the “ [symbol] ” example on the screen. The user needs to confirm that the password is the same as the password card, and then the password card is taken out. So the calibration is completed and the galactose test can be carried out.

(2) Galactose Detection

The user first washes and completely wipes dry the fingers, and then and put the biological sampling needle into the biological sampling device at the fixed place. After inserting a galactose test strip into the test strip slot of the meter, the meter is automatically activated, and shows “ [symbol] ”, example on the screen. The user confirms that the password on the screen is the same as that on the test strip box, and may sample biological sample when a blood drop symbol “ [symbol] ” flashes on the screen.

Before sampling biological sample, wipe the fingertips with alcohol cotton. After the fingertips are completely dry, biological samples are obtained by using a biological sampling device to lightly prick the fingertips. By enabling the biological sample to lightly touch a biological sample absorption opening of the test strip, the test strip would automatically absorb the biological sample to a reaction zone. When seeing that a transparent test window in the test strip reaction zone completely appears red and hearing a “beep” sound, the fingertip biological sample can be moved. At the end of the test (after about 1 minute), a galactose value will be displayed on the screen. In addition, the potential of this readout can be transmitted to the others including medical practitioners through Bluetooth or similar connection through a mobile phone or computer.

After the test is completed, the test strip is taken out and discarded properly. If no test is performed consecutively, then the meter will be automatically deactivated after three minutes.

### Embodiment 2: Detection System Principle and Test

The present invention mainly provides a system for measuring galactose content in biological sample. Users take aforementioned galactose composition beforehand. After the galactose composition is metabolized by liver in the human body, the galactose or the metabolites thereof will presence in the blood. The users take a blood sample from fingertips, and drop the sample on the test strip which is claimed by the present invention. Due to the enzyme in the test strip, the enzyme could react with the galactose or metabolites thereof, then generate electric current through electrochemical reaction. Insert the test strip into the meter of the present invention, the meter detect the amount of galactose in the human body by detecting the electric current signal in the test strip. The users could thereby supervise the health condition of the users. Because the process of the detection is very simple, it could reduce the time of detecting galactose compared to the prior arts with high accuracy and precision.

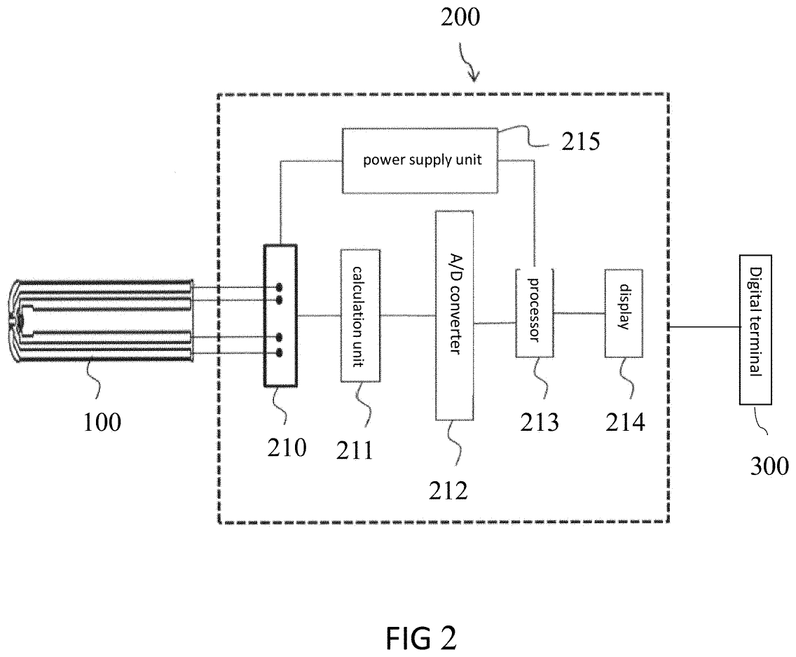

FIG. 2 is a block schematic view of the galactose detection system according to one embodiment of the present invention. The system comprises a test strip 100 and a meter 200. The meter 200 includes a connector 210 connected to the external, a calculation unit 211 for calculating concentration, an A/D converter 212, a processor 213, and a display 214. When the power supply unit 215 applies a signal(the signal is preferably a square wave signal at the frequency of 1 kHz-22 kHz; the voltage is 50 mV-5V, preferably 300 mV-800 mV) to the test strip via the connector 210, the galactose or metabolite thereof in biological sample and a enzyme in the test strip react through electrochemical reaction, producing a electrochemical information. The signal reacting with the electrochemical information produce a corresponding response signal, and the corresponding response signal is transmitted to the calculation unit 211 of the meter 200 via the connector 210. Then, the calculation units 211 will calculate the corresponding response signal, outputting the corresponding response signal to the A/D converter 212 to transform the corresponding response signal to a digital reaction signal which is further processed by the processor 213 and the measurement result will be displayed via the display 214. Furthermore, the digital reaction signal could be transmitted to a digital terminal 300, such as sending the signal of galactose concentration to mobile phone or computer through Bluetooth or wireless.

2-1 Accuracy Test

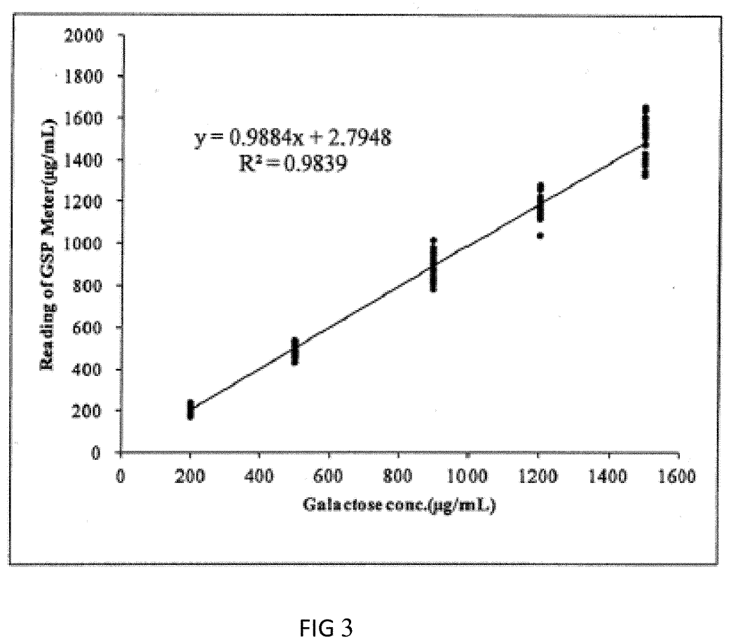

First, preparing five different concentrations of galactose samples (which are respectively 200 μg/ml, 500 μg/ml, 900 μg/ml, 1200 μg/ml and 1500 μg/ml), each taking 24 groups, and adding venous blood into them, then using the meter of the present invention to test the concentration values, calculating their average (μg/ml), standard deviation (S.D.) and coefficient of variations (% C.V.), and making a regression analysis chart, wherein the detection environment is room temperature (25±5° C.) and the relative humidity is 20-60%, as shown in FIG. 3. The reading value of the meter of the present invention has a high correlation coefficient of up to 0.98 of the actual galactose concentration, which represents highly accurate for the meter of the invention.

2-2 Precision Test

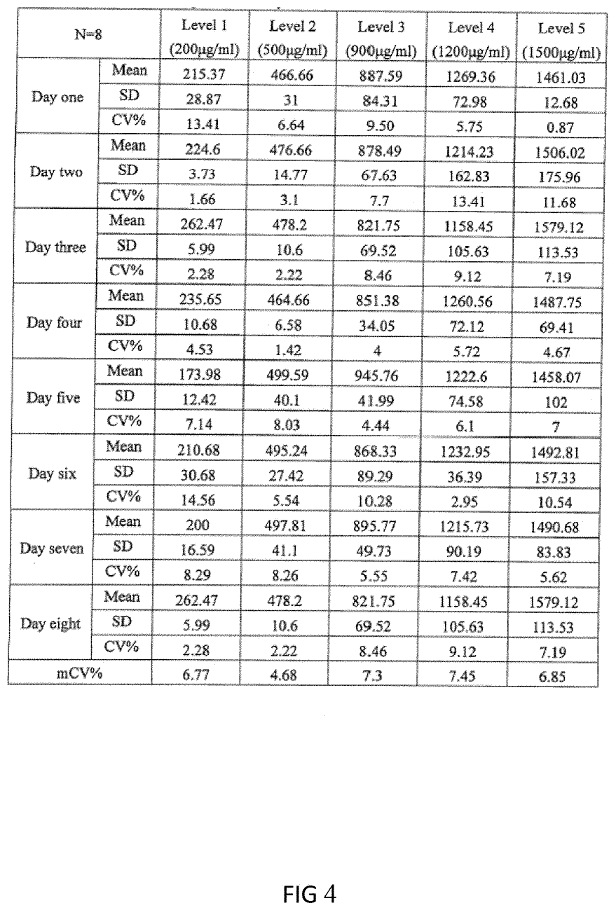

First, preparing five different concentrations of galactose samples (which are respectively 200 μg/ml, 500 μg/ml, 900 μg/ml, 1200 μg/ml and 1500 μg/ml) at a room temperature (25±5° C.) and 20-60% of relative humidity, each taking 3 groups, and adding venous blood into them, then using the meter of the present invention to test the concentration values and repeating the tests for eight days, calculating their average of the coefficient of variation (% C.V.) (as shown in FIG. 4). From the data in FIG. 4 shows that the average coefficient of variation (% C.V.) of the five samples in eight days ranged from 6.5-7.5 represents the high precision of the test instrument.

In light of foregoing result, the procedure of the galactose detection system of present invention is simple and rapid. It is because the formula of the galactose composition of the present invention can be metabolized rapidly by the liver in the human body, allowing the blood or body fluid contain galactose or metabolites thereof. Then, take the sample by fingertips. After the sample react with the enzyme in the test strip via electrochemical reaction, take the meter to detect the galactose for only 1 minute without preparing test specimen additionally. The procedure deduct the amount of steps to detect galactose which further reduce the detecting time. Therefore, the present invention provide a rapid, simple and highly accurate detecting galactose method for patients who need to detect galactose.

### Embodiment 3: Test Strip Detection

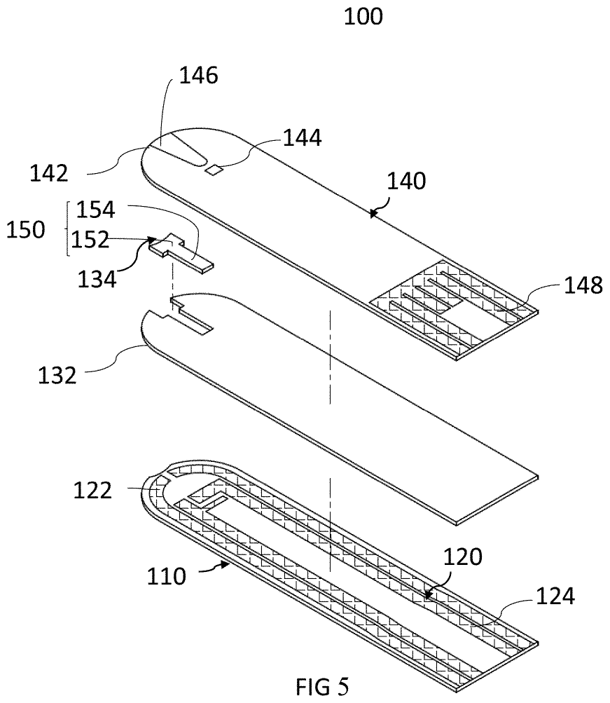

FIG. 5 is a schematic view of the test strip according to one embodiment of the present invention. The test strip 100 includes an insulating substrate 110, an electrode unit 120, a first insulating spacer 130 and a second insulating spacer 140. The test strip contains enzyme reacting with galactose or metabolites thereof to have electrochemical reaction.

In this embodiment, the insulating substrate 110 has a flat surface which has electrical insulation and heat resistance between 40-120° C. The material of the insulating substrate 110 is selected from polyvinyl chloride (PVC), glass fiber (FR-4), polyester suphone, bakelite plate, polyethylene terephthalate (PET), polycarbonate (PC), polypropylene (PP), polyethylene (PE), polystyrene (PS), glass plate, ceramic or any combination of the above-described materials.

As shown in FIG. 5, the electrode unit 120 is configured on the insulating substrate 110. The electrode unit 120 comprises a first end 122 and a second end 124 which are opposite. In the present embodiment, the electrode unit 120 may be composed of a plurality of electrodes insulated from each other. The material of the electrode unit 120 can be any conductive substance, such as palladium glue, platinum glue, gold glue, titanium glue, carbon glue, silver glue, copper glue, gold-silver mixed glue, carbon-silver mixed glue, or any combination of the above-described conductive materials. In one embodiment, the electrode unit 120 consists of a conductive carbon powder layer or a metal layer. In still another embodiment, the electrode unit 120 consists of a conductive adhesive silver layer and a conductive carbon powder layer thereon, wherein the impedance of the conductive carbon powder layer is generally much greater than that of the conductive silver adhesive layer or other metal layers.

The materials of the first insulating spacer 130 may include but not limited to the polyvinyl chloride (PVC) insulating adhesive tape, ethylene terephthalic acid ester insulating adhesive tape, heat dried insulating varnish, or ultraviolet light cured insulating varnish. The first insulating spacer 130 covers a part of the electrode unit 120 (namely the part of the first end 122), and includes a reaction zone channel 134 located on a first edge 132 of the first insulating spacer 130. The first end 122 is exposed to the reaction zone channel 134. The sample (for example, blood) is suitable for filling the reaction zone channel 134 to perform the subsequent electrochemical reaction. The two long sides of the reaction zone channel 134 are ladder shaped, and the width of the reaction zone channel 134 adjacent to the first edge 132 is greater than the width away from the first edge 132.

The reaction zone channel 134 has at least one reaction layer 150 covering at least one electrode unit 120 in the reaction zone channel 134 and containing at least one galactose and a conductive medium, with samples (such as blood) to generate chemical reaction. The reaction layer 150 can further include a galactose enzyme measuring area and a conductive medium measuring area.

The composition of the reaction layer 150 can be but is not limited to the enzyme, coenzyme, conductive medium, buffer solution, stabilizer and surfactant. Wherein the conductive medium is used to receive the electrons generated after an active substance is reacted with the sample, conducts the electrons to the meter 200 via the electrode unit 120, and includes but is not limited to: ferrocene, ferrocenium, methylene blue, tris(acetonitrile)ruthenium trichloride, 2,5-dihydroxybenzoquinone, phenazinemethosulfate, tetrathiafulvalene, tetra-cyano-quino-dimethane, methyl viologen, toluidine blue, 5,6-diamino-1,10-phenanthroline, [M(bpy)3]2+(M=Ru or Os; BPY=2,2′-bipyridine). In addition, the conductive medium could be a metallic ion compound, wherein the metallic ion compound includes but is not limited to MgCl₂, BeCl₂, CaCl₂, SrCl₂, BaCl₂ or a combination thereof which can be dissolved in an aqueous solution in a metallic ion manner under the absorption action between electrons and charges; the buffer solution includes but is not limited to neutral and alkaline buffer solutions of Tris, Tris-HCl, PBS, MES, CHES, Borate, Universal buffer mixtures (CPB), MOPS, TES, HEPES, TAPSO, Tricine, Bicine and TAPS; The stabilizer comprises but is not limited to Xylitol, mannitol, polyxylose, araboxylan, mannan, trehalose, PEG, PVA, PEO, Methocel, agarose, sol-gel, collagen, chitosan, BSA, casein, neo protein, amino acid or any one combination thereof; The surfactant includes but is not limited to a cationic surfactant, an anionic surfactant, a neutral ionic surfactant, and a nonionic surfactant.

In the present embodiment, the second insulating spacer 140 covers the first insulating spacer 130, a part of the electrode unit 120 and a part of the insulating substrate 110. Since the second insulating spacer 140 completely covers the reaction zone channel 134 of the first insulating spacer 130, the upper, lower, left and right surfaces of the reaction zone channel 134 are enclosed by three wall surfaces of the second insulating spacer 140, an insulating substrate 110 and the first insulating spacer 130 beside the reaction zone channel 134 to form a pentahedral enclosed pipe. When the sample enters the reaction zone channel 134 via a biological sampling opening, the adhesive force of the biological sample in the reaction zone channel 134 is greater than the cohesive force of the biological sample, such that the biological sample can go forward persistently.

In the present embodiment, the first edge 132 of the first insulating spacer 130, the second edge 142 of the second insulating spacer 140, and the same side edge of the insulating substrate 110, as a whole, are all in a convex arc shape. In addition, as shown in FIG. 5, the edge of the insulating substrate 110 concaves inwards relative to the front half part of the reaction zone channel 134. The test strip 100 of the present embodiment utilizes the convex tip of the second edge 142 of the second insulating spacer 140 and the concave structure of the insulating substrate relative to the front half part of the reaction zone channel 134 to reduce the cohesive force of the biological sample, and enables the biological sample to go forward rapidly under the action of capillary phenomenon. In addition, in the present embodiment, the second insulating spacer 140 further comprises a vent hole 144 located at a position away from the second edge 142, namely at the end of the reaction zone channel 134 of the first insulating spacer 130. The vent hole 144 is used to discharge the air in the reaction zone channel 134, in case the biological sample is blocked by an air bubble and cannot go forward smoothly in the reaction zone channel 134.

Due to the instability of the enzyme protein, the enzyme cannot be preserved in an alkaline environment or dry condition. Therefore, the enzyme is generally stored in an acidic solution, such as preserved in acidic amine sulphate solution with a very short storage time. The enzyme will lose activity once becoming dry, so the enzyme cannot be stored in solid state. However, the test strip in the present invention with the above formula and structure allow the enzyme not only to be preserved in an acidic environment, but also be solidified and stored in a neutral or alkaline environment. Furthermore, the enzyme with the formula can retain activity in a dry state and can be preserved for a long time. Therefore, the invention has broken through the previous restrictions to ensure that the enzyme can be solidified and dried which is effective to dry the enzyme on the test strip and still remain active.

3-1 Detection of Test Strip Detectable Volume

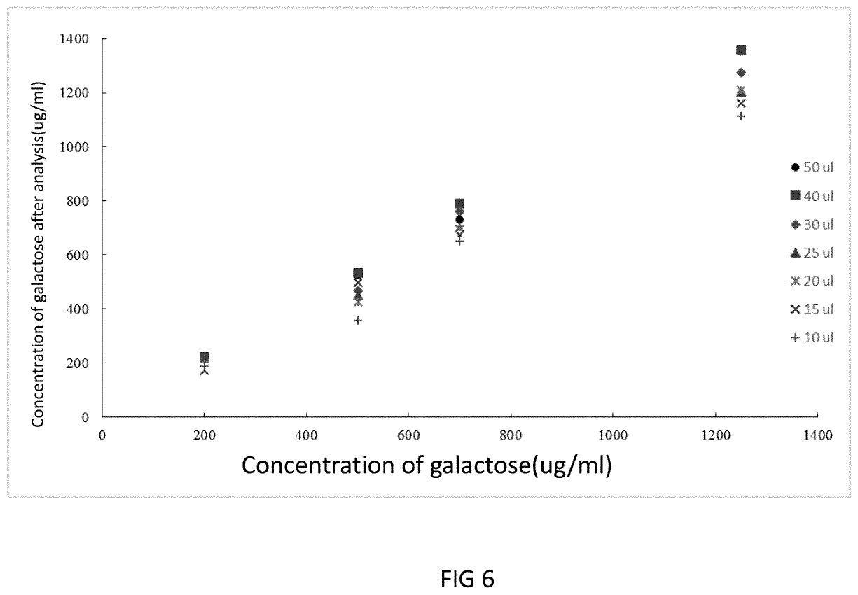

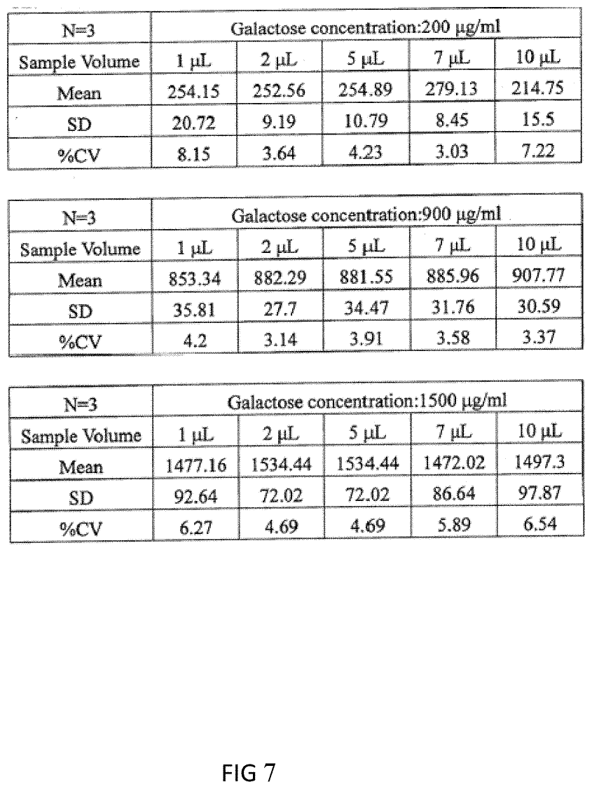

FIG. 6 is a biological volume analysis of a general filter paper. The result shows that at least 30 μl fingertip biological sample volume of the filter paper can be used to ensure that the error is less than 15%. However, the test strip of the present invention can achieve a small volume biological sample detection. The experiment method is to prepare three different concentrations of galactose samples (which are 200 μg/ml, 900 μg/ml, and 1500 μg/ml, respectively), each of the galactose samples will be 1, 2, 5, 7 and 10 μl volume to detect the data values (see FIG. 7) and, repeat each of the detections three times, and then calculate the average (μg/ml), standard deviation (S.D.) and coefficient of variation (% C.V.), wherein the acceptable average C.V value of galactose samples below the concentration of 250 μg/ml or less needs less than 20%, while the acceptable average C.V of galactose samples in the range of 251-1500 μg/ml needs less than 15%. FIG. 7 shows that the average C.V value of galactose samples with the concentration of 200 μg/ml in each volume is in the range of 3.03-8.15% which is less than 15%, while the averages C.V value of galactose samples with the concentrations of 900 μg/ml and 1500 μg/ml in each volume are both in the range of 3.14-6.54% which is less than 20%. Therefore, the test strip of the present invention can detect the galactose with the volume greater than or equal to 1 μl.

3-2 Test of Test Strip Long Term Stability

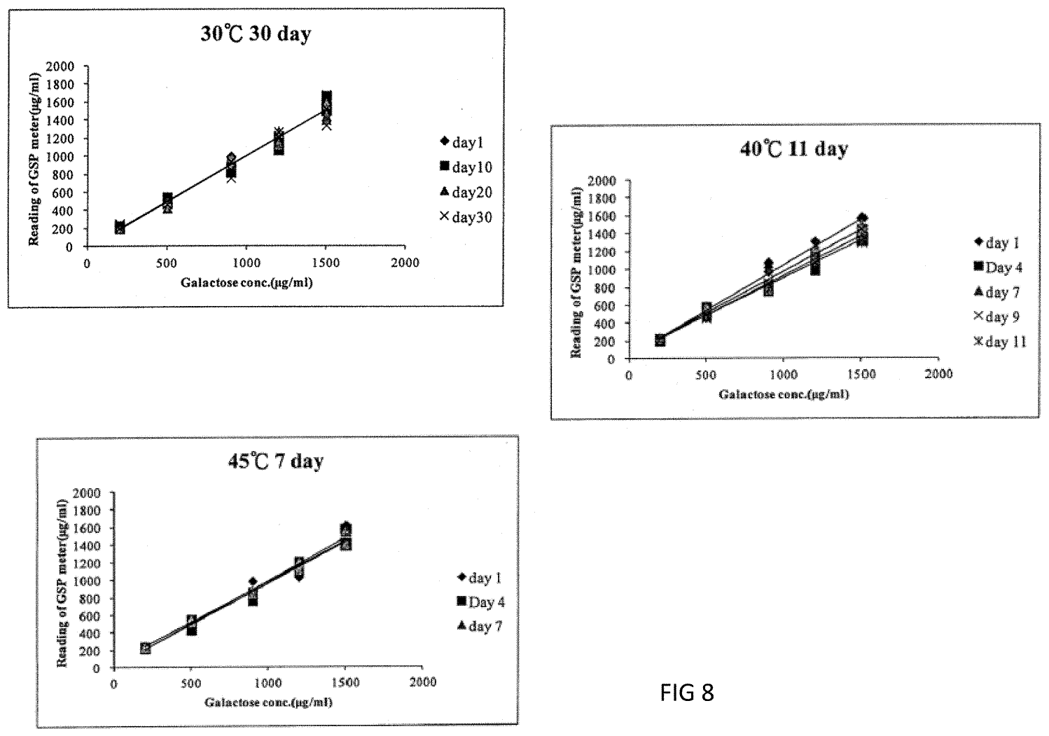

To evaluate the service condition of the test strip under the severe environment, the preservation days is estimated in a 4° C. environment. Five different concentrations of galactose samples (which are 200 μg/ml, 500 μg/ml, 1200 μg/ml, 900 μg/ml and 1500 μg/ml, respectively) were prepared, and they were divided into three groups of 30° C., 40° C. and 45° C., respectively, and then the reading value of galactose was measured one by one, wherein the acceptable average C.V value of galactose below the concentration of 250 μg/ml is less than 20%, while the acceptable average C.V. value of galactose in the range of 251-1500 μg/ml needs less than 15%, and the correlation coefficient (R) should be greater than 0.9. According to the results of FIG. 8, the test strip of the present invention can be stored at 4° C. for 545.32 days (longest), 30° C. for 30 days, 40° C. for 11 days, and 45° C. for 7 days. The preferred storage environment for the test strip of the present invention is 4° C.-10° C. It can be seen that the test strip is now stable for 180 days at 4° C., and for 60 days at a room temperature. It is estimated that the test strip of the present invention can remain stable for up to 545 days stored at 4° C. by the acceleration test.

3-3 Hematocrit Evaluation Test

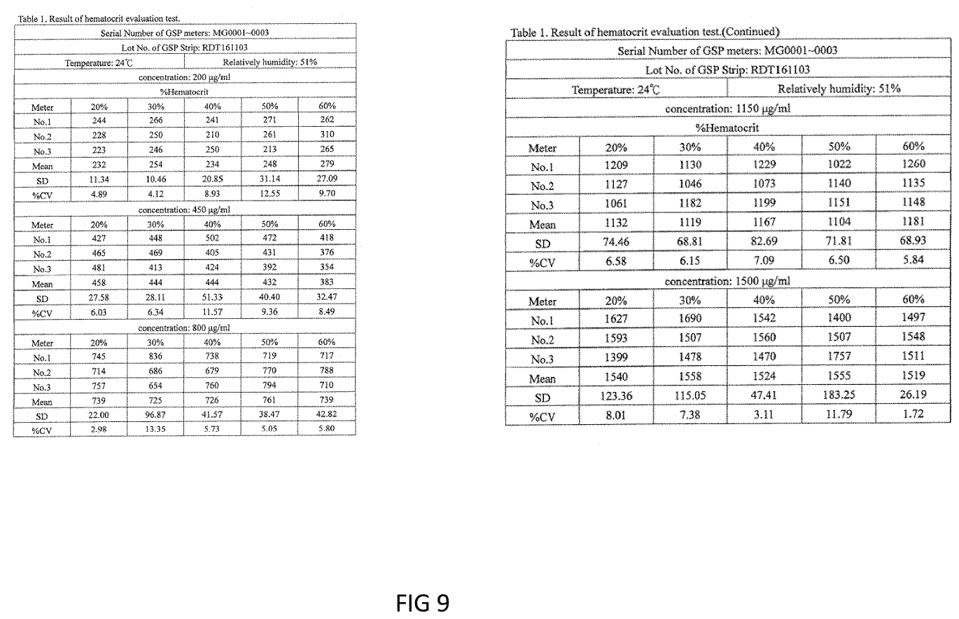

In order to evaluate whether the test strip can detect the different hematocrits (HCT) of samples in a normal range, five different concentrations of galactose biological samples (200 μg/ml, 450 μg/ml, 800 μg/ml, 1150 μg/ml and 1500μg/ml, respectively) were prepared, and each HCT sample of 20%, 30%, 40%, 50% and 60% were prepared. The reading values of galactose were then measured one by one. Among them, the acceptable average C.V value of galactose below the concentration of 250 μg/ml needs less than 20%, while the acceptable average C.V value of galactose in the range of 251-1500 μg/ml needs less than 15%, and the correlation coefficient (R) should be greater than 0.9. As shown in FIG. 9, the average C.V value of galactose in the range of 450-1500 μg/ml is less than 15%, and the average C.V value of galactose with the concentration of 200 μg/ml is less than 20%. Therefore, the test strip of the present invention can at least detect the biological sample in the HCT range of 20%-60%.

3-4 Repeatability Test

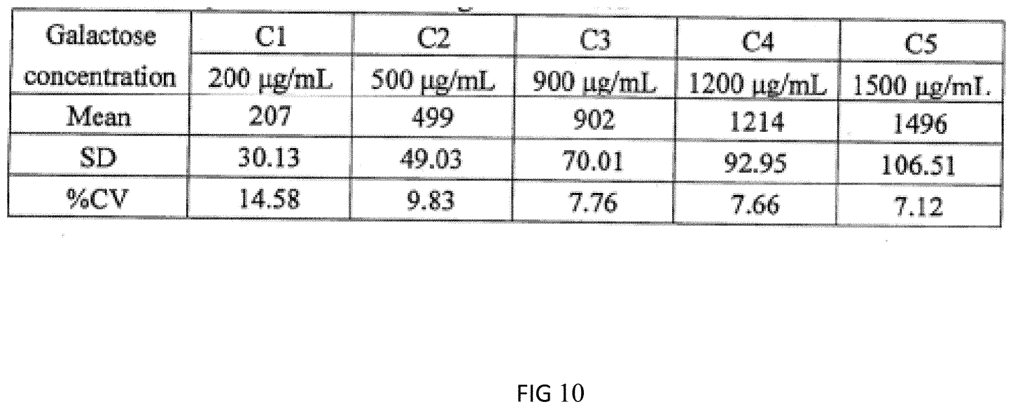

In order to evaluate whether the test result of the galactose rapid quantitative detection system is repeatable, a repeatability test is performed as follows: five different concentrations of galactose samples (200 μg/ml, 450 μg/ml, 900 μg/ml, 1200 μg/ml and 1500 μg/ml, respectively) were prepared to add into the biological samples, wherein each concentration will be tested by three meters, and each meter will repeat the test six times. The acceptable average C.V value of galactose below the concentration of 250 μg/ml needs less than 20%, and the acceptable average C.V value of galactose in the range 251-1500 μg/ml needs less than 15%. From the result of FIG. 10, the average C.V value of galactose samples in the range of 500-1500 μg/ml is in the range of 7.12-9.83% which is less than 15%; and average C.V of galactose samples with the concentration of 200 μg/ml is 14.58% which is less than 20%. Therefore, the test result of the galactose rapid quantitative detection system of the present invention is repeatable.

In light of foregoing result, the test strip of the present invention can detect the 1 μL volume of the biological sample at the minimum. Due to aforementioned enzyme and formula, the test strip can be stored for 60 days at room temperature, for 180 days at 4° C. It overcome the obstacle of preserving problem. In addition, because the minimal volume of biological sample is 1 μL which avoid discomfort caused by large wound per test, while maintaining high accuracy of test results. The present invention provide the users a preferred tool for detecting galactose.

### Embodiment 4: Using the Detection System to Determine Liver Function

4-1 Comparison Between Oral Administration Galactose OGSP Result and Intravenous Injection Galactose GSP Result

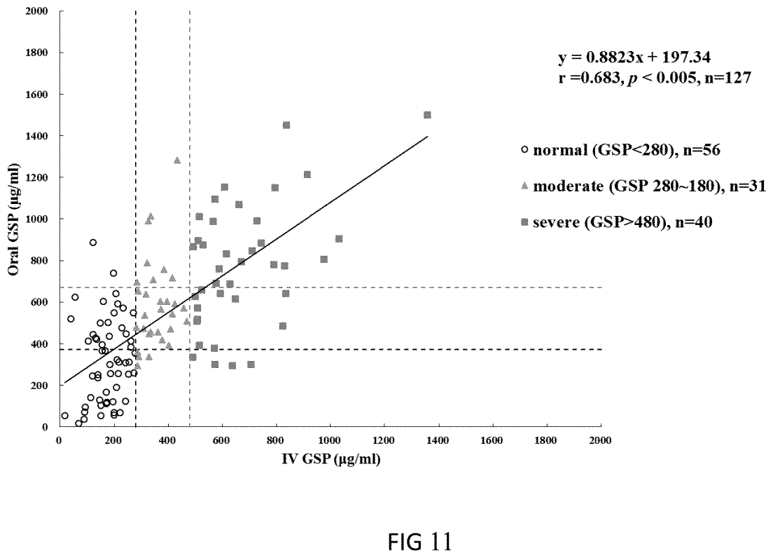

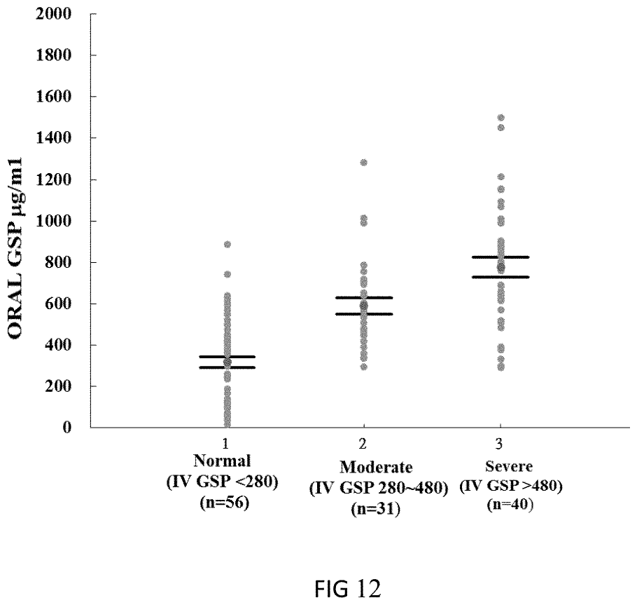

As shown in FIGS. 11 and 12, a total of 127 subjects (56 subjects have normal liver function and 71 subjects have impaired liver function) are tested to determine the correlation between an intravenous injection galactose GSP result and an oral administration galactose OGSP result. As suggested in Digestion 1992, 52:222-231, the subjects joining the intravenous injection galactose GSP test are divided into three groups: the subjects with the GSP less than 280 μg/ml are defined in a liver function normal group; the subjects with the GSP in the range of 280-480 μg/ml are defined in a liver function moderately impaired group; and the subjects with the GSP greater than 480 μg/ml are defined in a liver function severely impaired group. From the results of FIGS. 10 and 11, the oral administration galactose OGSP value is higher than the intravenous injection galactose GSP value and the oral administration galactose OGSP value grows with the impairment degree of the liver function, wherein the OGSP and the GSP are positively correlated. The oral administration galactose OGSP values of the subjects in the liver function normal group are in the range 318±27 μg/ml (average±standard error) with the minimum value 18 μg/ml and maximum value 887 μg/ml. The oral administration galactose OGSP values of the subjects in the liver function mildly or moderately impaired group are in the range 590±40 μg/ml with the minimum value 294 μg/ml and maximum value 1282 μg/ml. The oral administration galactose OGSP values of the subjects in the liver function severely impaired group are in the range 777±48 μg/ml with the minimum value 293 μg/ml and maximum value 1499 μg/ml. Table 5 shows the intravenous injection galactose GSP results and the oral administration galactose OGSP results of the three groups of subjects that the oral administration galactose OGSP value grows with the impairment degree of the liver function. Particularly, the oral administration galactose OGSP value is higher than the intravenous injection galactose GSP value. From FIGS. 11, 12 and Table 5, it can be determined that the oral administration galactose OGSP values of the subjects in the liver function normal group are mainly in the range of 264-372 μg/ml (average±2*standard error) and the oral administration galactose OGSP values of the subjects in the liver function mildly or moderately impaired group are mainly in the range of 510-670 μg/ml. The oral administration galactose OGSP values of the subjects in the liver function severely impaired group are mainly in the range of 681-873 μg/ml (average±2*standard error). Even if the results of the subjects are varied due to individual difference, the oral administration galactose OGSP values of the subjects in the liver function normal group generally do not exceed 670 μg/ml, and the OGSP values of the subjects in the liver function impaired groups are generally greater than 370 μg/ml. Therefore, further liver function tests should be taken in the subjects whose OGSP value is greater than 370 μg/ml. In additional to intraveneous injection, similar results were obtained by other injection or other administration ways.

Table 1 The intravenous injection galactose GSP result and oral administration galactose OGSP result of the subjects (average±standard error)

| | | Mild or moderate | Severe |
|---|---|---|---|
| | Normal | impairment | impairment |
| | liver function | of liver | of liver function |
| | (N = 56) | function (N = 31) | (N = 40) |
| IV GSP(μg/ml) | 247 ± 16.5*** | 423 ± 26.0*** | 630 ± 41.0*** |
| Digestion 1992; | | | |
| 52: 222-231 | | | |
| IV GSP (μg/ml) | 174 ± 8*** | 359 ± 10*** | 667 ± 29*** |
| OGSP(μg/ml) | 318 ± 27*** | 590 ± 40*** | 777 ± 48*** |

***P < 0.005 (ANOVA & LSD analysis)

Embodiment 5: Neonatal Galactosemia Screening

Galactosemia is a hereditary disease which is attributed to the fact that there is not enough galactose clastic enzyme in the patient, so that galactose accumulates in the body. This results in the symptoms of sleepiness, emesis, diarrhea, incapability of normal growth, jaundice, and the like. Through newborn screening, one can be sure there will be no adverse effects in infants breast milk. The galactose meter of the present invention can be used for the screening of neonatal galactosemia. The test neonatal galactosemia screening does not rely on protein or lactose digestion, but adopts a first biological sample of infants, so the galactose composition is not required to be taken before the screening and biological sample is sampled from a toe tip. If the galactose value of the biological sample is detected to be greater than 100 μg/ml, which represents the risk of neonatal galactosemia in the newborn, and further examination is needed.

Embodiment 6: Semiautomatic Arm Operation Analysis

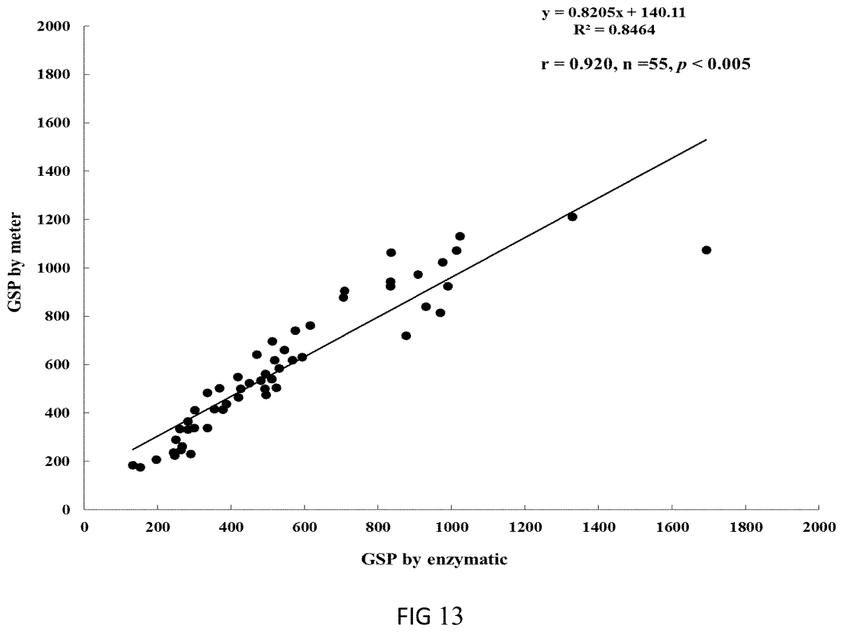

FIG. 13 shows a comparison between conventional filter paper enzyme analysis and the enzyme analysis of the galactose rapid quantitative detection system performed by using a semiautomatic robotic arm with a galactose single-point method. The analysis is separated into intravenous injection galactose GSP and oral administration galactose OGSP, wherein the correlation coefficient of conventional filter paper enzyme analysis and the enzyme analysis of the galactose rapid quantitative detection system of intravenous injection galactose GSP is 0.963, and the correlation coefficient of oral administration galactose OGSP is 0.927. In conclusion, both intravenous injection galactose GSP and oral administration galactose OGSP have high correlation coefficient above 0.9. Therefore, the galactose rapid detection system of the present invention can be produced through large scale production.

In summary, the galactose rapid quantitative detection system provided by the invention has already been tested by accuracy and precision, can be used to detect liver functions and examine galactose related diseases, such as neonatal screening for galactosemia, and can determine the physical state of medical staffs or patients to then judge whether a further examination is required.

What is claimed is:

1. A galactose rapid quantitative detection system, comprising:
a galactose composition including a galactose, a buffer and an 0˜99% antioxidant, which enters a body and after metabolism by the liver and produces a biological sample;
a test strip or a filter paper, comprising an enzyme, the enzyme would react with the biological sample producing a electrochemical information; and
a meter including:
a power supply unit for providing a signal;
a connector for receiving the signal provided by the power supply unit and transmitting the signal to the test strip or the filter paper, wherein the signal reacting with the electrochemical information produce a corresponding response signal, and the connector transmit the corresponding response signal to the meter;
a calculation unit for calculating the corresponding response signal;
an A/D convertor for receiving the corresponding response signal from the calculation unit, transforming the corresponding response signal calculated by the calculation unit into a digital reaction signal;
a processor for processing the digital reaction signal;
a display for displaying the digital reaction signal; and
a digital terminal for receiving the digital reaction signal.

2. The system according to claim 1, wherein the buffer is selected from a group consisting of ascorbic acid buffer, citrate buffer, phosphate buffer, acetate buffer, carbonate buffer, and triethanolamine buffer.

3. The system according to claim 1, wherein the antioxidant is selected from a group consisting of vitamin C or/and sodium bisulfite, vitamin A, vitamin E, flavonoids, polyphenols, Ethylenediaminetetraacetic acid(EDTA), Diethylenetriaminepentaacetic acid (DTPA), NTA-Nitrilotriacetate acid (NTA).

4. The system according to claim 1, wherein the galactose includes D-(+)-galactose, L-(−)-galactose, stable isotope galactose, cyclic galactose or galactose derivative.

5. The system according to claim 1, wherein the galactose composition is administrated through oral administration, injection, spray, inhalation, buccal, rectal, suppository or other medical acceptable way.

6. The system according to claim 5, wherein the way of oral administration is to let users take the galactose composition in advance, then the content of galactose in the body is measured by measuring the content of galactose in the biological sample.

7. The system according to claim 5, wherein the way of injection is to let users inject the galactose composition into the body in advance, then the content of galactose in the body is measured by measuring the content of galactose in the biological sample.

8. A test strip according to claim 1, wherein the test strip comprises:
an insulating substrate,
an electrode unit configured on the insulating substrate,
a first insulating spacer covering a part of the electrode unit and including a reaction zone channel sited at a first edge of the insulating spacer, wherein another part of the electrode unit is exposed to the reaction zone channel; and
a second insulating spacer including a second edge, the second insulating spacer covering the reaction zone channel of the first insulating spacer, and the first edge of the first insulating spacer, the second edge of the second insulating spacer, and the same side edge of the insulating substrate are all in a convex arc shape, and the edge of the insulating substrate concaves inwards relative to the front half part of the reaction zone channel; wherein the reaction zone channel comprises at least a reaction layer, the reaction layer is covered by the electrode unit in the reaction zone channel including at least galactose and a conductive medium to react with biological sample through electrochemical reaction;
wherein the test strip utilizes the convex tip of the second edge of the second insulating spacer and the concave structure of the insulating substrate relative to the front half part of the reaction zone channel to reduce the cohesive force of the biological sample, and enables the biological sample to go forward rapidly under the action of capillary phenomenon;
wherein the enzyme which oxidize, reduce, decompose or metabolize galactose.

9. The test strip according to claim 8, wherein the insulating substrate is selected from the group consisting of polyvinyl chloride (PVC), glass fiber (FR-4), polyester suphone, bakelite plate, polyethylene terephthalate (PET), polycarbonate (PC), polypropylene (PP), polyethylene (PE), polystyrene (PS), glass plate, ceramic or any combination thereof.

10. The test strip according to claim 8, wherein the electrode unit is selected from the group consisting of palladium, platinum, gold colloid, titanium, carbon, silver, copper, gold and silver.

11. The test strip according to claim 8, wherein the reaction layer is selected from the group consisting of enzyme, coenzyme, buffer solution, stabilizer and surfactant.

12. The test strip according to claim 8, wherein the conductive medium is selected from the group consisting of ferrocene, ferrocenium, methylene blue, tris(acetonitrile)ruthenium trichloride, dihydroxybenzoquinone, phenazinemethosulfate, tetrathiafulvalene tetra-cyano-quino-dimethane, methyl viologen, toluidine blue, 5,6-diamino-1,10-phenanthroline, 2,2′-bipyridine.

13. The test strip according to claim 8, wherein the conductive medium further compries metal ion compound, the metal ion compound is selected from the group consisting of MgCl₂, BeCl₂, CaCl₂, SrCl₂, BaCl₂ and any one combination thereof

14. The test strip according to claim 11, wherein the buffer solution is selected from the group consisting of Tris, Tris-HCl, PBS, MES, CHES, Borate, Universal buffer mixtures (CPB), MOPS, TES, HEPES, TAPSO, Tricine, Bicine and TAPS.

15. The test strip according to claim 11, wherein the stabilizer is selected from the group consisting of Xylitol, mannitol, polyxylose, araboxylan, mannan, trehalose, PEG, PVA, PEO, Methocel, agarose, sol-gel, collagen, chitosan, BSA, casein, neo protein, amino acid and any one combination thereof.

16. The test strip according to claim 11, wherein the surfactant is selected from the group consisting of a cationic surfactant, an anionic surfactant, a neutral ionic surfactant, and a nonionic surfactant.

17. The test strip according to claim 8, wherein the test range of galactose in the test strip is 50-2000 μg/ml.

18. The test strip according to claim 8, wherein the enzyme can be dried, solidified and stored in a neutral, acidic or alkaline environment.

19. A method of performing the system according to claim 1 within a user, comprising:
(1) The user takes a preparation with galactose in its composition in advance;
(2) A biological sample is obtained by using a biological sampling device;
(3) the biological sample is absorbed by a test strip from the biological sampling device;
(4) the test strip is inserted into a meter; and
(5) the user or a professional medical staff read the value of galactose concentration a disease or liver residual function of the user.

20. The method according to claim 19, wherein the method can be manipulated by the subject or professional staff

21. The method according to claim 19, wherein the disease is neonatal galactosemia.
